(19) United States
(12) Patent Application Publication
(10) Pub. No.: US 2020/0281908 A1
(43) Pub. Date: Sep. 10, 2020

(54) MEANS FOR TREATING DEMENTIA OF VARIOUS ETIOLOGY

(71) Applicant: Rakhimdzhan Ahmetdzhanovich ROZIEV, Obninsk (RU)

(72) Inventors: Anna Yakovlevna GONCHAROVA, Obninsk (RU); Kenes Tagaevich ERIMBETOV, Borovsk (RU); Ekaterina Valeryevna BONDARENKO, Balabanovo (RU); Ksenija Jur'evna VLASOVA, Obninsk (RU)

(21) Appl. No.: 16/464,283

(22) PCT Filed: Nov. 28, 2017
(86) PCT No.: PCT/RU2017/050122

§ 371 Date: May 27, 2019

(30) Foreign Application Priority Data
Nov. 28, 2016 (RU) 2016146594

(51) Int. Cl. A61K 31/44; A61K 9/127; A61P 25/28

(52) U.S. Cl. CPC A61K 31/44; A61P 25/28; A61K 9/127

(57) ABSTRACT

The invention relates to medicine, specifically to pharmacology, and concerns a new liposomal composition on the basis of methyl-4-[2-oxo-1,2-bis[[2-(4-pyridinyl)ethyl]amino]ethyl]-benzoate succinate as a targeted delivery means for the treatment of dementia of various etiology. The claimed composition is obtained by the film method followed by hydration and ultrasonication.

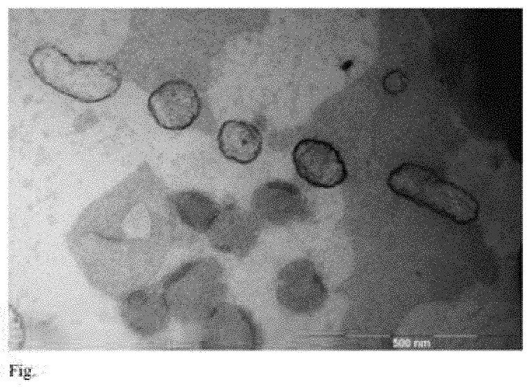

The invention relates to medicine, to pharmacology in particularly, and relates to the use of the liposomal formulation of methyl 4-[2-oxo-1,2-bis[[2-(4-pyridinyl)ethyl]amino]ethyl]benzoate succinate as an agent with targeted delivery for the treatment of dementia of various origin.

Currently, dementia (dementia) in the elderly, which is manifested in 4% of the population aged 65 years and at least 35% of the population aged 85 years, is an important medical challenge. The number of patients suffering from dementia, due to the increase in life expectancy, is growing rapidly and is expected to reach 80 million people in 2040. Alzheimer's disease (AD) is reported to be the most common form of dementia, about 60% of all cases. It is a progressive, irreversible neurodegenerative disease with under-investigated pathogenesis and with little to no effective treatment. Life expectancy is constantly increasing, with the most rapidly growing part of the total over-65 population. Consequently, the incidence of diseases associated with age, such as dementia and others, can be expected to increase significantly, and nootropics will be more and more in demand. Such agents have been actively developed for many years, but so far the main focus of the search remains on an attempt to pharmacologically correct cholinergic deficiency, which has led to the development of a number of well-known drugs based on acetylcholinesterase inhibitors (Tacrine, Amiridin, Aricept, etc.), which yet still have a very limited clinical applicability. Memantine, an NMDA receptor antagonist, is also widely used now. It leads to a short-term improvement in cognitive functions with little to no prevention of AD progression.

There is a known method, compound, and pharmaceutical formulation and drug for the restoration of lost memory under normal and under pathological conditions/Russian Patent No. 2457205, C07D401/12, 27 Jul. 2012/

It is proposed to use heterocyclic low-molecular sAPP-mimetics and their pharmaceutical composition as an agent for restoring lost memory under normal and pathological conditions/Russian Patent No. 2465273, C07D401/12, 27 Oct. 2012/. The said compounds can be used to restore memory lost, for example, as a result of a neurodegenerative disease or other central nervous system diseases. Such diseases can include Alzheimer's disease, Parkinson's disease; Huntington's disease (chorea); multiple sclerosis; cerebellar degeneration; amyotrophic lateral sclerosis; dementia with Lewy bodies; spinal muscular atrophy; peripheral neuropathy; spongiform encephalopathy; HIV-associated dementia; multi-infarct dementia; frontotemporal dementia; leukoencephalopathy; chronic neurodegenerative diseases; stroke, cerebral ischemic, reperfusion and hypoxic injury; epilepsy; cerebral ischemia; glaucoma; Down syndrome; encephalomyelitis; meningitis; encephalitis; neuroblastoma; schizophrenia, depression; neurodegenerative processes.

The above said drugs for the restoration of lost memory under normal and under pathological conditions are not characterized by targeted delivery, are not effective enough when used in low doses, which is a technical problem. Reducing the effective doses of drugs for patients suffering from dementia of various origin is a topical problem. Since for this category of patients, ease of administration and dosage of drugs are by no means unimportant, in connection with their long-term (life) prescription.

To solve the indicated technical problem and achieve a technical result, namely the reduction of the therapeutic doses administrated, the present invention proposes a targeted-delivery agent for the treatment of dementia of various origin, which is a liposomal formulation of methyl 4-[2-oxo-1,2-bis[[2-(4-pyridinyl)ethyl]amino]ethyl]benzoate succinate, obtained by the film method, followed by hydration and sonication.

Liposomes are known to be membrane-enclosed vesicles spontaneously formed in mixtures of phospholipids with water, the wall of which consists of one or more phospholipid bilayers (layers of two molecules thick) in which other substances are embedded, such as proteins. There is water or solution inside liposomes. Liposomes are successfully used as drug vehicles since their chemical composition is similar to natural cell membranes; they are universal, which allows them to deliver a wide range of reagents; they do not provoke allergic reactions. However, there are a number of difficulties in the use of liposomes in medicine. First, liposomes are absorbed by the cells of the reticuloendothelial system, with more of them found in the liver, spleen, bone marrow, lymph nodes, and blood flow. Therefore, drug delivery using liposomes to other organs and body systems is more difficult. Secondly, lipoproteins, while exchanging lipids with liposomes, contribute to the destruction of liposomes and the outflow of their contents. In addition to these disadvantages of liposomes as a dosage form, there are difficulties associated with the high rate of absorption of liposomes by macrophages, which leads to a short-term therapeutic effect of the drug. To prevent rapid capture of liposomes by macrophages, a method of surface stabilization with polyethyleneglycol was developed

The introduction 1,2-gasterol-sn-glycero-3-phosphoethylamine-N-carboxy (polyethyleneglycol)-2000 into the lipid membranes increases the circulation time of the composition in the blood flow and prevents rapid discharge of the active drug from the liposomes. Another disadvantage of nanocontainers is the difficulty of passage across the blood-brain barrier (BBB) and the delivery of the active substance to the target organ. To solve these limitations, a vector is introduced onto the surface of the container (in this case, liposomes), which due to the affinity for the body's transport system or receptors on the cell surface delivers the active substance in the composition to the desired target organ.

The claimed liposomal form of methyl 4-[2-oxo-1,2-bis[[2-(4-pyridinyl)ethyl]amino]ethyl]benzoate succinate was prepared as follows. Liposomal formulation was produced by the film method, followed by hydration. The required set of lipids (phosphatidylcholine, 1,2-deterol-sn-glycero-3-phosphoethylamine-N-carboxy (polyethylene glycol)-2000) and cholesterol were dissolved in chloroform in a round-bottomed flask. The solvent was then evaporated on a rotary evaporator to obtain a film, the dissolution/evaporation procedure was repeated until a homogeneous film was obtained. The resulting film was hydrated with a solution of methyl 4-[2-oxo-1,2-bis[[2-(4-pyridinyl)ethyl]amino]ethyl]benzoate succinate in phosphate buffer (pH=7.4: 10 mM). The resulting suspension was treated sonicated for 5 minutes and then put through an extruder with the required pore size (0.4 and 0.2 μm) to obtain a homogeneous liposome suspension. To separate the liposomes from the free preparation, the suspension was put through a NAP-25 column.

The surface of the formulation was then modified for vector (targeted) delivery. As a vector for drug delivery to the brain and its retention, the LAT1 transport system was used, which proved to be effective for delivering L-dopa to treat Parkinson's disease. The ammo acid leucine was covalently immobilized onto the surface of liposomes to bind liposomes to LAT1 transport agents. Carboxyl groups were present on the surface of the bilayer and they were activated with N-nitroxy-sulfosuccinimide and 1-ethyl-3-(3-dimethylaminopropyl) carbodiimide reagents. The excess activating reagents were disposed of using a NAP-25 column. Further, leucine was added to the activated liposomes and incubated for 30 min. Then free leucine was disposed of using a column for the separation of low molecular weight substances.

When modifying the surface of liposomes with leucine as a vector, the size increases, and the surface potential does not change much. This is due to the fact that amino groups are bound in the leucine, and carboxyl groups remain free on the surface. For the initial stage of studying the effect of PEG and leucine in the composition of liposomes, the FIGURE shows a micrograph of the liposomal form of methyl 4-[2-oxo-1,2-bis[[2-(4-pyridinyl)ethyl]amino]ethyl]benzoate succinate made by transmission electron microscopy (TEM:

According to TEM, the liposome size is 160-200 nm. The oblong shape may be caused by aggregation during test sample preparation. Inside the liposomes, 5 nm small particles, which are the crystalline form of the active substance, were also found. Immobilization of leucine on the surface results in no significant changes in size, since the molecule itself is small, and there is no jump in the surface charge of liposomes, due to the small amount of the vector introduced onto the surface (no more than 4%) and the presence of a free amino group

Antioxidant dihydroquercetin was added to the liposomes for their storage. The suspension was stored at 4° C. The main characteristics of the liposomal formulation and its composition are shown in Tables 1 and 2.

TABLE 1
Main characteristics of liposomal formulation

| Characteristics | Values | |
|---|---|---|
| Concentration of methyl 4-[2-oxo-1,2-bis[[2-(4- | 0.6-1.2 | g/L |
| pyridinyl)ethyl]amino]ethyl]benzoate succinate | | |
| in liposomal suspension | | |
| Total lipids | 12 | g/L |
| Degree of drug inclusion | 5-10% | |
| Liposome size | 150-200 | nm |

| | |
|---|---|
| Polydispersity index | 0.15-0.20 |

TABLE 2
Composition of the liposomal formulation

| Components | Content, w % |
|---|---|
| methyl 4-[2-oxo-1,2-bis[[2-(4-pyridinyl) | 5-10 |
| ethyl)amino]ethyl]benzoate succinate | |
| Phosphatidylcholine | 70-80 |
| Cholesterol | 1-4 |
| DSPE-PEG | 2-4 |
| Leucine | 1-2 |

The nature of the invention is illustrated by examples of specific implementation and a FIGURE, which shows Micrograph of the liposomal form of methyl 4-[2-oxo-1,2-bis[[2-(4-pyridinyl)ethyl]amino]ethyl]benzoate succinate made by transmission electron microscopy (TEM). A prototype of the claimed drug for the treatment of dementia of various origin is methyl 4-[2-oxo-1,2-bis[[2-(4-pyridinyl)ethyl]amino]ethyl]benzoate succinate shown in Patents No. 2465273 and No. 2457205.

Example 1. The aim of the study is to assess the effect of the liposomal formulation of methyl 4-[2-oxo-1,2-bis[[2-(4-pyridinyl)ethyl]amino]ethyl]benzoate succinate on conditioned passive avoidance reflex (CPAR) amnesia caused by maximal electroshock seizure (MES) in comparison with the pharmaceutical substance of this compound and piracetam in rats.

### Study Procedure

CPAR is a basic method for studying the effects of drugs affecting memory in any manner whatsoever [9]. When exposed to CPAR, it is possible to identify both memory improvement and its deterioration (amnesic effect). CPAR is based on the inborn hole exploratory behaviour of rodents—the tendency to hide in a limited darkened space.

The apparatus for studying the action of drugs is a dark chamber with an electrode floor. The dark chamber is connected through a square guillotine door with a hinged platform. The hinged platform is illuminated by a 60 watt lamp. The dark chamber is located oil the laboratory table, and the platform hangs above the floor at a height of 800 mm.

First, CPAR was developed in rats. Rats were placed on a lighted platform in from of the dark chamber of the apparatus with their tail toward the inlet and recorded latent time of the reflex. Then, in the dark chamber, the rat received a single 10-second pain stimulation with an electric current (0.3 mA) through the electrode floor (training)

The training recall and CPAR amnesia test was performed 24 hours after the training and was carried out when the animal was placed into the apparatus for a second time with the registration of the 3-minute latent period of the first entry of the rat into the dark experimental chamber, where it had previously received pain stimulation, and the number of animals (in %) that did not go into the dark compartment of the chamber.

Amnesia was invoked by MES Electric stimulus (250 V, 115-120 mA, 50 Hz, for 0.1 sec) for obtaining MES was applied to the rat through special cornal (ocular) electrodes, previously soaked in saline MES was performed directly after CPAR training.

Experimental groups. The animals were divided into 7 arms:
Arm I: control without amnesia (saline).
Arm II: control with amnesia (MES)
Arm III: Prototype 0.25 mg/kg+MES.
Arm IV: Prototype 1.0 mg/kg+MES.
Arm V: Current liposomal formulation 0.25 mg/kg+MES.
Arm VI: Current liposomal formulation 1.0 mg/kg+MES.
Arm VII: Piracetam 400 mg/kg+MES.

The prototype and the current liposomal composition were administered intragastrically. Piracetam was administered once intraperitoneally 40 minutes before the experiment

### Study Results

Control without amnesia. When placed on an illuminated platform, rats with a short latent period go into the dark chamber and receive pain stimulation through the electrode floor (training). During the reflex recalling test 24 hours after the training, the rats remember the trained reflex, and when they are placed on the illuminated platform, the animals stay there for the main experiment time and do not go into the dark dangerous chamber, where they have received pain stimulation (training) the day before (Table 1).

Control with amnesia. MES performed directly after training causes amnesia, which is expressed in a statistically significant (P≤0.05) substantial reduction (2.5 times) of the latent period of going into the dark chamber when recalling CRAP and a significant (P≤0.05) decrease (2.0 times) in the number of animals that did not go into the dark compartment of the camera during the recalling test, i.e. that remember the pain stimulation received there the day before. Only 37.5% of animals after MES remember the electric shock, compared to control (without MES) animals, in which this finding was 100% (Table 1).

The data obtained indicate a pronounced amnesic effect of MES.

The effect of the current liposomal formulation. At doses of 0.25 and 1.0 mg/kg, the current liposomal formulation was found to increase the duration of the latent period of entry into the dark chamber during the CPAR recalling test, dramatically and statistically significantly (P≤0.05), and increase the number of animals without amnesia that did not go into the dark compartment of the camera at all during the recalling test, i.e. that remember the pain stimulation received there the day before (Table 1), statistically significantly (P≤0.05). The data obtained indicate the ability of the current liposomal formulation, especially at a dose of 0.25 mg/kg, to reduce the amnesia caused by MES dramatically, statistically significantly, as compared with the control with amnesia, and the prototype at a dose of 0.25 mg/kg.

TABLE 1
Study of the effect of the test drugs at doses of 0.25 and 1.0 mg/kg (once, intragastrically)
and Piracetam at a dose of 400 mg/kg (once, intraperitoneally) on CPAR amnesia caused by MES
(M ± m, n = 10)

| | | CPAR recalling test | |
|---|---|---|---|
| | | 24 hours later: | |
| | CPAR training | | Number of animals that did |
| | Latent time of | Latent time of the | not go into the dark |
| Animal groups | the reflex, sec | reflex, sec | chamber, % |
| Control without amnesia | 21.88 ± 4.08 | 180.00 ± 0.00 | 100 |
| Control with amnesia | 26.94 ± 7.17 | 76.06 ± 20.21* | 37.5 [symbol] |
| MES | | | |
| Prototype 0.25 mg/kg [symbol] | 20.38 ± 4.38 | 107.86 ± 15.12 | 48.5 [symbol] |
| MES | | | |
| Prototype 1 mg/kg + MES | 13.29 ± 2.78 | 118.13 ± 17.26** | 57 [symbol] |
| Current liposomal | 19.23 ± 3.25 | 152.13 ± 12.26*** | 83.5^{&&} |
| formulation 0.25 mg/kg [symbol] | | | |
| MES | | | |
| Current liposomal | 14.75 ± 3.01 | 131.86 ± 19.12** | 75.0 [symbol] |
| formulation 1 mg/kg [symbol] | | | |
| MES | | | |
| Piracetam 400 mg/kg [symbol] | 19.11 ± 5.28 | 97.17 ± 21.43 | 50 |
| MES | | | |

*significance of differences from the control without amnesia (saline), with P ≤ 0.05 (Student's t-test);
**significance of differences from the control with amnesia (MES), with P ≤ 0.05 (Student's t-test);
***significance of differences from the prototype of 0.25 mg/kg with amnesia (MES), with P ≤ 0.05 (Student's t-test);
^{#}significance of differences from control (saline), with P ≤ 0.05 (Fisher's test)
^{&}significance of differences from control with MES, with P ≤ 0.05 (χ²-test)
^{&&}significance of differences from the prototype of 0.25 mg/kg with MES, with P ≤ 0.05 (χ² test)
Piracetam effect: Piracetam at a dose of 400 mg/kg (once, intraperitoneally) does not significantly change the duration of the latent period of entry into the dark chamber during the CPAR recalling test and does not increase the number of animals without amnesia that did not go into the dark compartment of the camera at all during the recalling test, i.e. that remember the pain stimulation received there the day before (Table 1), significantly. The data obtained indicate that Piracetam at a dose of 400 mg/kg does not have antiamnesic effect in the MES-induced amnesia test. [symbol]
indicates data missing or illegible when filed

Thus, the current liposomal formulation at doses of 0.25 and 1.0 mg/kg (once, intragastrically) has a pronounced antiamnesic effect in a stringent basic MES-induced CPAR amnesia test. The effect of the current liposomal formulation was found for all the recorded parameters and is expressed in a statistically significant increase in both the duration of the latent period of entry into the dark chamber and the number of animals that did not go into the dark compartment of the chamber during the CPAR recalling test. The current liposomal formulation at doses of 0.25 and 1.0 mg/kg has approximately the same antiamnesic effect, which suggests that it has a wide range of antiamnesic effect. The best results were obtained when the current liposomal formulation was administered to rats at a dose of 0.25 mg/kg under the conditions of MES-induced amnesia.

In terms of the severity of the antiamnesic effect in the MES-induced CPAR amnesia test, the current liposomal formulation at doses of 0.25 and 1.0 mg/kg exceeds Piracetam (400 mg/kg), since it increases both the duration of the latent period of entry into the dark chamber and the number animals that did not go into the dark compartment of the chamber, during the CPAR recalling test, significantly, while Piracetam at a dose of 400 mg/kg does not have an antiamnesic effect on any of the parameters.

Example 2. The aim of the study is to assess the specific pharmacological activity of the liposomal formulation of methyl 4-[2-oxo-1,2-bis[[2-(4-pyridinyl)ethyl]amino]ethyl]benzoate succinate in comparison with the pharmaceutical substance of this compound and memantine on memory loss when modelling AD using neurotoxin streptozotocin in rats.

### Study Procedure

In this study, the modelling of AD was performed using the procedure recommended in the Methodological Recommendations for the preclinical study of nootropic drugs [9, 12].

According to the procedure, AD modeling was performed by introducing streptozotocin toxin into the cerebral ventricles of the rat.

The surgical neurotoxin injection into the brain was performed in anaesthetized animals (with chloral hydrate at a dose of 200 mg/kg, ip) in a stereotaxic apparatus.

First, the cranial trepanation was performed in animals according to the stereotaxic coordinates [24]; AP (1.0), L (1.5-2). Streptozotocin at a dose of 3 mg/kg was injected bilaterally into the lateral ventricles of the brain according to the stereotaxic coordinates: AP (1.0): L (1.5-2): H (3.0). The toxin was injected at a rate of 0.1 μl per minute; the injected solution volume was 5 μl in each ventricle

The cranial trepanation of the control (sham-operated) animals was performed according to the stereotaxic coordinates: AP (1.0): L (1.5-2), and distilled water was then injected bilaterally into the lateral ventricles of the brain also according to the stereotaxic coordinates, in the same volume and at the same rate as the studied drugs.

1 hour after surgery, the intragastric administration of substances to rats started, which continued for 14 days.

The test pharmaceutical substance (Prototype) and the current liposomal formulation of this compound were administered to rats in 2 doses of 0.25 mg/kg and 1 mg/kg. Memantine at a dose of 1 mg/kg. Distilled water was administered to control animals.

The day after the last injection of substances, procedures that allow testing the training and memory of animals started.

Evaluation of the antiamnesic effect of PAM-12 in comparison with memantine was performed using the CPAR method [9]. A detailed description of the CPAR method is given in Example 1. The CPAR recalling test was performed 24 hours and 14 days after the training. To do this, the rat was placed on the platform with its tail toward the hole in the darkened compartment, and the latent period of entry of the animal therein was recorded (maximum observation time of 180 seconds), and the number of animals that did not go into the dark dangerous compartment and remained on the illuminated hanging platform (rats without amnesia, well remembering the situation) was also reported. Memory loss was determined in the terms of the reduction of the latent time of the CPAR and increase in the number of animals that went into the dark chamber during reflex recalling test, compared to that of the control

### Animal Groups

The animals were randomly divided into 8 arms.

1. Intact control—distilled water, intragastrically (i.g.), 14 days-12 rats.

2. Control—sham-operated animals—distilled water, i.g. 14 days-12 rats.

3. Active control—injection of streptozotocin (STZ) 3 mg/kg into the cerebral ventricles+distilled water, i.g., 14 days-12 rats

4. Prototype 0.25 mg/kg, i.g., 14 days: the first injection 1 hour after the injection of STZ into the cerebral ventricles and then daily for 14 days-12 rats.

5. Prototype 1.0 mg/kg, i.g., 14 days: the first injection 1 hour after the injection of STZ into the cerebral ventricles and then daily for 14 days-12 rats.

6. Current liposomal formulation 0.25 mg/kg, i.g., 14 days: the first injection 1 hour after the injection of STZ into the cerebral ventricles and then daily for 14 days-12 rats

7. Current liposomal formulation 1.0 mg/kg, i.g., 14 days: the first injection 1 hour after the injection of STZ into the cerebral ventricles and then daily for 14 days-12 rats.

8. Memantine 1 mg/kg. i.g., 14 days: the first injection 1 hour after the injection of STZ into the cerebral ventricles and then daily for 14 days-12 rats.

### Study Results

It was found that in the course of CPAR training, the animals of all groups did not differ from each other in terms of the latent time of entry into the dark chamber (Table 2). The data obtained indicate non-effect of both toxin and drugs on the orienting-exploratory motor activity of rats and no hole exploratory behavior disturbance under the action of intracerebral injection of STZ.

The behavior of sham-operated animals that underwent the brain surgery procedure, but did not receive the toxin, did not differ from the behavioral indices of intact animals in the CPAR training and recalling test 24 hours, 7 and 14 days after training (Tables 2 and 3).

Rats, which STZ was injected, (active control) were found to suffer from distinct cognitive deficits. The results obtained during the CPAR recalling test 24 hours, 7 and 14 days after the training in rats, which STZ was injected showed learning disorders, memory loss and severe amnesia in animals. During the reflex recalling test, the rats forgot about the pain stimulation received in the dark compartment and went therein within a short latent period.

The process of forgetting had a temporary dynamics. In that way, 42% of the rats did not remember the acquired skill 24 hours later, 58%—7 days later, and 66.7%—14 days later (Table 3).

Severe amnesia in rats, which streptozotocin was injected, is also indicated by a 1.4-time (tendency), 1.5-time (p<0.05) and 2.2-times (p<0.05) reduction of the latent period of entry of rats into the dark chamber during PCAR recalling test 24 hours, 7 and 14 days later, respectively, compared with the sham-operated animals (Table 2).

The current liposomal formulation at a dose of 0.25 mg/kg (i.g.) showed pronounced antiamnesic activity in the CPAR test in rats on an AD model, induced by the injection of STA into the cerebral ventricles.

During the CPAR recalling test 24 hours after the training, the current liposomal formulation at a dose of 0.25 mg/kg increased the latent time of entry into the dark dangerous chamber significantly. 1.47 and 1.35 times compared with the active control and 0, 25 mg/kg prototype groups, respectively (Table 2).

During the CPAR recalling test 7 days after the training, the current liposomal formulation at a dose of 0.25 mg/kg increased the latent time of entry into the dark dangerous chamber significantly. 1.50 and 1.37 times compared with the active control and 0, 25 mg/kg prototype groups, respectively (Table 2).

TABLE 2
Effect of the test drugs vs. memantine on cognitive deficiency under the conditions of
CPAR test on a streptozotocin-induced rat model of AD in terms of the latent period (M ± m, n = 12).

| | Latent period of going into the dark chamber, sec | | | |
|---|---|---|---|---|
| | | Recalling I.P;. | | |
| | Training LP₁, | 24 hours | 7 days later, | 14 days |
| Substance, dose | sec | later, sec | sec | later, sec |
| Intact control | 17.9 ± 2.3 | 167.7 ± 12.3 | 167.7 ± 12.3 | 153.5 ± 17.9 |
| distilled water, i.g. | | | | |
| Sham-operated, distilled water, i.g. | 20.8 ± 3.1 | 169.3 ± 10.7 | 167.0 ± 10.7 | 156.3 ± 16.1 |
| Active control | 30.4 ± 9.1 | 122.6 ± 14.9 | 110.3 ± 18.3* | 75.7 ± 19.3* |
| Distilled water, i.g. + STZ | | | | |
| Prototype 0.25 mg/kg, i.g [symbol] STZ | 24.3 ± 5.1 | 133.1 ± 13.3 | 121.3 ± 17.8 | 102.8 ± 19.4 |
| Prototype 1.0 mg/kg, i.g. [symbol] STZ | 30.1 ± 12.1 | 147.7 ± 16.2 | 132.3 ± 2.8 | 124.9 ± 20.3 |
| Current liposomal formulation 0.25 | 17.3 ± 3.7 | 180.0 ± 12.3## | 165.8 ± 13.2## | 147.1 ± 15.6## |
| mg/kg, i.g. [symbol] STZ | | | | |
| Current liposomal formulation 1.0 | 18.8 ± 4.2 [symbol] | 158.2 ± 15.7# | 145.3 ± 16.8# | 137.8 ± 18.4# |
| mg/kg, i.g. [symbol] STZ | | | | |
| Memantine 1 mg/kg, i.g. [symbol] | 20.4 ± 3.4 | 166.4 ± 13.6 | 166.4 ± 13.6# | 143.5 ± 19.2# |
| STZ | | | | |

Notes:
*p < 0.05, the significance of differences with respect to the Mann-Whitney test relative to the group of sham-operated animals.
#p < 0.05, the significance of differences with respect to the Mann-Whitney test relative to the Active Control group (injection of streptozotocin)
##p < 0.05, the significance of differences with respect to the Mann-Whitney test relative to the Prototype 0.25 mg/kg group (injection of streptozotocin)
LP₁-the latent period of the first entry into the dark chamber during training.
LP₂-the latent period of entry into the dark chamber during CPAR recalling test.
During the CPAR recalling test 14 days after the training, the current liposomal formulation at a dose of 0.25 mg/kg increased the latent time of entry into the dark dangerous chamber significantly, 1.68 and 1.43 times compared with the active control and 0, 25 mg/kg prototype groups, respectively (Table 2). [symbol]
indicates data missing or illegible when filed

TABLE 3
Effect of the test drugs vs memantine on cognitive deficiency under the conditions of
CPAR test on a streptozotocin-induced rat model of AD in terms of the percentage of rats with
reflex impairment (M ± m, n = 12)

| | Number of animals with amnesia that went into the dark chamber | | |
|---|---|---|---|
| | during the CPAR recalling test/Number of animals in the group | | |
| Substance, dose | 24 hours later, sec | 7 days later, sec | 14 days later, sec |
| Intact control | 1/12 (8.3%) | 1/12 (8.3%) | 2/12 (16.7%) |
| Distilled water, p.o. | | | |
| Sham-operated | 1/12 (8.3%) | 2/12 (16.7%) | 2/12 (16.7%) |
| Distilled water, p.o. | | | |
| Active control, | 6/12 (50.0%)* | 7/12 (58.3%) | 8/12 (66.7%)* |
| STZ 3 mg/kg | | | |
| Prototype 0.25 mg/kg, i.g. + | 4/12 (33.3%) | 5/12 (41.7%) | 6/12 (50%) |
| STZ | | | |
| Prototype 1.0 mg/kg, i.g. + | 3/12 (25%) | 4/12 (33.3%) | 5/12 (41.7%) |
| STZ | | | |
| Current liposomal | 0/12 (0%)## | 1/2 (8.3%)## | 2/12 (16.7%)## |
| formulation 0.25 mg/kg, | | | |
| i.g. [symbol] STZ | | | |
| Current liposomal | 1/12 (8.3%)# | 2/12 (16.7%)# | 3/12 (25%)# |
| formulation 1.0 mg/kg, i.g. [symbol] | | | |
| STZ | | | |
| STZ [symbol] | 1/12 (8.3%)# | 1/12 (8.3%)# | 3/12 (25%)# |
| Memantine 1 mg/kg, p.o | | | |

Notes:
*p < 0.05, the significance of differences with respect to the Mann-Whitney test relative to the group of sham-operated animals.
#p < 0.05, the significance of differences with respect to the Fisher's test relative to the Active Control group (injection of streptozotocin).
##p < 0.05, the significance of differences with respect to the Fisher's test relative to the Active Control group (injection of streptozotocin). [symbol]
indicates data missing or illegible when filed

In the course of treatment with the current liposomal formulation at a dose of 0.25 mg/kg, 100% of the group animals (p<0.05) remembered the pain stimulation and did not go into the dark chamber 24 hours after the CPAR training, 91.7%—7 days later (p<0.05), and 83.3%—14 days later (Table 3)

The data obtained indicate that, on day 14 after the CPAR training, the positive effect of the liposomal formulation at a dose of 0.25 mg/kg on cognitive deficit caused by streptozotocin remained.

During the CPAR recalling test 24 hours, 7 and 14 days after the training, the current liposomal formulation at a dose of 1.0 mg/kg (i.g.) increased the latent period of entry into the dark chamber in rats with AD compared with the Active Control (Table 2) statistically significantly.

After the CPAR training in the course of treatment with the current liposomal formulation at a dose of 1.0 mg/kg, the latent time of entry of rats with AD into the dark chamber was longer than in 0.25 and 1.0 mg/kg prototype groups, but these changes were of a trend nature.

In the course of treatment with the current liposomal formulation at a dose of 1.0 mg/kg, 91.7%, 83.3% and 75.0% of the animals remembered the pain stimulation and did not go into the dark chamber 24 hours, 7 and 14 days after the CPAR training, respectively (Table 3). The differences with the active controls (injection of STZ) in terms of the percentage of animals with CPAR amnesia was statistically significant. However, in terms of the percentage of animals with CPAR amnesia, the differences with the prototype at a dose of 1.0 mg/kg were not statistically significant. Thus, the best results were obtained when the current liposomal formulation was administered to rats at a dose of 0.25 mg/kg under the conditions of STZ-induced AD model.

Memantine at a dose of 1 mg/kg had a pronounced antiamnesic effect, eliminating the cognitive deficit in rats of the streptozotocin-induced AD model.

The drug was effective in all test periods 24 hours, 7 and 14 days after the training. Thus, in the course of treatment with memantine, the latent time of going into the dark chamber was longer than in the control, but statistically insignificant 24 hours later, and the latent period was 1.5 and 1.9 times statistically significantly longer than in the Active control group 7 and 14 days later (Table 2). Administration of memantine to rats with STZ-induced AD caused a significant decrease in the number of animals that went into the dark chamber during the CPAR recalling test. In the course of treatment with the formulation, the number of rats who forgot about the pain stimulation 24 hours, 7 and 14 days after the CPAR training was 5, 7 and 2.7 times less (statistically significant), respectively, compared to rats of the Active Control group (Table 3).

The comparison of the antiamnesic effects of the current liposomal formulation and memantine suggests that the drugs have a similar activity in their ability to eliminate the cognitive deficit that occurs in rats when modelling AD by intraventricular injection of neurotoxin STZ. Moreover, the current liposomal formulation has a more pronounced effect on the cognitive deficit than memantine when it is evaluated 24 hours after the training.

Thus, as a result of the studies performed, it was found that the current liposomal formulation eliminates cognitive deficits in rats of an STZ-induced AD model in the best possible way. The dependence of the antiamnesic effect of the current liposomal formulation on the injected dose level was found. The maximum effect of the current liposomal formulation has at a dose of 0.25 mg/kg, and with an increase in the dose to 1 mg/kg, the antiamnestic effect decreases slightly.

1. An agent with targeted delivery for the treatment of dementia of various origin, which is a liposomal formulation of methyl 4-[2-oxo-1,2-bis[[2-(4-pyridinyl)ethyl]amino]ethyl]benzoate succinate obtained by the film method, followed by hydration and sonication.
